(19) United States
(12) Patent Application Publication
(10) Pub. No.: US 2008/0226546 A1
(43) Pub. Date: Sep. 18, 2008

(54) Kit for preparation of nano-targeted liposome drug in combined radionuclide therapy and chemotherapy

(76) Inventors: Te-Wei Lee, Taoyuan (TW); Ya-Jen Chang, Taoyuan (TW); Liang-Cheng Chen, Taoyuan (TW); Chia-Yu Yu, Taoyuan (TW); Shu-Pei Chiu, Taoyuan (TW); Shu-Ling Chen, Taoyuan (TW); Tsai-Yueh Luo, Taoyuan (TW); Chih-Hsien Chang, Miolie (TW); Gann Ting, (US)

Correspondence Address: MICHAEL LIN, 5F 79 Roosevelt Rd. Sec. 2, TAIPEI, 106, TW

(21) Appl. No.: 11/687,650

(22) Filed: Mar. 18, 2007

(51) Int. Cl. A61K 51/12; A61K 51/00; A61P 35/00; A61K 51/04

(52) U.S. Cl. 424/1.21; 424/1.61; 424/1.65

(57) ABSTRACT

This invention is to manufacture a kit for preparation of nano-targeted liposome drugs in combined chemotherapy and radionuclide therapy. It is a kit consisting of three components: (1) A 10 ml vial A which contains BMEDA, gluconate acetate, SnCl₂. (2) A 10 ml vial B which contains DSPC, cholesterol, DSPE-PEG, and Doxorubicin(DXR) (or Daunorubicin, Vinolbine). (3) A 10 ml vial C which contains ¹⁸⁸ReO₄⁻ (or ¹⁸⁶ReO₄⁻) solution. The procedure of using the kit is as follows: (1) Remove the contents of the ¹⁸⁸ReO₄⁻ (or ¹⁸⁶ReO₄⁻) solution from vial C. (2) Inject the ¹⁸⁸ReO₄⁻ (or ¹⁸⁶ReO₄⁻) solution into the vial A, and the mixtures react in appropriate temperature. (3) Remove the contents of the ¹⁸⁸Re-BMEDA (or ¹⁸⁶Re-BMEDA) solution from vial A. (4) Inject the ¹⁸⁸Re-BMEDA (or ¹⁸⁶Re-BMEDA) solution into the vial B, and the mixtures react in appropriate temperature. The reconstituted solution in the vial B is applied to combine bimodality radiochemotherapy for treatment of tumor and ascites.

### BACKGROUND OF THE INVENTION

1. Field of the Invention

This invention relates to manufacture a kit for the preparation of nano-targeted liposome drugs and the kit is applicable to combine targeted radionuclide therapy and chemotherapy for imaging and treatment of tumor and ascites.

2. The Prior Art

Liposomes, which are biodegradable and essentially non-toxic vehicles, can encapsulate both hydrophilic and hydrophobic drugs. In addition, liposomes can be used to carry radioactive compound as payloads. Liposomes can provide several advantages for bimodality radiochemotherapy for the following reasons:

Biocompatibility: Lipid and cholesterol used for liposome manufacture are common constitutes of cell membranes and therefore are easily metabolized. Enhanced permeability and retention (EPR) effect: Due to the unregulated tumor growth and location of endothelial lining in angiogenetic vasculature, the blood vessels in tumors have a tendency to leak, which induces the spontaneous accumulation of liposomes from blood circulation into the tumor. This phenomenon of concentration and localization of drugs in tumor tissues is called the enhanced permeability and retention effect. In addition, angiogenesis is the major mechanism of ascites fluid production.

Varying uniform sizes: Liposome with variable homogeneous particle size ranges can readily be produced by using the extrusion techniques.

There are two diagnostic and therapeutic radionuclides, ¹⁸⁸Re and 186Re, which have excellent physical properties. The physical characteristics of ¹⁸⁸Re and ¹⁸⁶Re are listed in Table 1. Bao et al. have developed a direct labeling method using ^{99m}Tc-BMEDA complex to label the commercially available pegylated liposome doxorubicin. (J. Pharmacol Exp Ther, 308: 419-425, 2004). However, the product of kit for the preparation of ¹⁸⁸Re-(or ¹⁸⁶Re) BMEDA/DXR-Liposome and application in the treatment of tumor and ascites has not been found yet.

TABLE 1
Physical Characteristics of ¹⁸⁸Re and ¹⁸⁶Re Radionuclides.

| | | | | | β-ray | | | |
|---|---|---|---|---|---|---|---|---|
| | Physical | | γ-ray | | | | Range in | |
| Radio- | half-life | Mode of | Energy | Abundance | Energy (MeV) | | tissue(mm) | |
| nuclide | (T½) | decay | (MeV) | (%) | Max. | Ave. | Max. | Ave. |
| ¹⁸⁸Re | 16.98 h | β⁻(100) | 0.155 | 14.9 | 2.12 | 0.765 | 11 | 3.5 |
| ¹⁸⁶Re | 3.8 d | β⁻(92) | 0.139 | 9 | 1.075 | 0.323 | 3.6 | 1.8 |
| | | EC (8) | | | | | | |

### SUMMARY OF THE INVENTION

The main object of the present invention is to provide a kit for preparation of nano-targeted liposome drugs in combined chemotherapy and radionuclide therapy. It is a kit consisting of three components: (1) A 10 ml vial A which contains BMEDA, gluconate acetate, SnCl₂. (2) A 10 ml vial B which contains DSPC, cholesterol, DSPE-PEG, and Doxorubicin (or Daunorubicin, Vinolbine). (3) A 10 ml vial C which contains ¹⁸⁸ReO₄⁻ (or ¹⁸⁶ReO₄⁻) solution. The procedure of using the kit is as follows: (1) Remove the contents of the ¹⁸⁸ReO₄⁻ (or ¹⁸⁶ReO₄⁻) solution from vial C. (2) Inject the ¹⁸⁸ReO₄⁻ (or ¹⁸⁶ReO₄⁻) solution into the vial A, and the mixtures react in appropriate temperature. (3) Remove the contents of the ¹⁸⁸Re-BMEDA (or ¹⁸⁶Re-BMEDA) solution from vial A. (4) Inject the ¹⁸⁸Re-BMEDA (or ¹⁸⁶Re-BMEDA) solution into the vial B, and the mixtures react in appropriate temperature. The reconstituted solution in the vial B is applied to combine targeted radionuclide therapy and chemotherapy for treatment of tumor and ascites.

The product of kit in this invention for preparation of nano-targeted liposome drugs in combined bimodality radiochemotherapy has proved to be more simple, convenient, effective and easier than the prior art is.

### BRIEF DESCRIPTION OF THE DRAWINGS

FIG. 1 shows targeting and nuclear imaging of tumors with prepared passive nano-targeted Re-188 radionuclides encapsulated in liposomes;

FIG. 2 and FIG. 3 indicate the effect of drugs treatment upon the tumor volume and the survival ratio after drugs treatment, respectively, and

FIG. 4 indicates mice receiving i.p. injection of ¹⁸⁸Re-BMEDA /DXR-Liposome with its survival ratio.

### DETAILED DESCRIPTION OF THE PREFERRED EMBODIMENTS

The following abbreviations are employed in the description of the examples:

BMEDA: N,N-bis(2-mercaptoethyl)-N′,N′-diethylethylenediamine

DSPC: Distearoyl phosphatidylcholine

PEG: Polyethylene glycol

DSPE: Distearyl phosphatidylethanolamine

### EXAMPLE 1

### The Preparation of Vial A Component

5 mg of BMEDA and 0.5 mL of 0.17 mol/L glucohepatonate dissolved in 10% acetate solution were added into vial A. Then, flushing with N2 gas for 1 minutes, followed by the addition of 120 μL (10 μg/μL) of stannous chloride. After flushing with N2 gas, the vial A was sealed.

### EXAMPLE 2

### The Preparation of Vial B Component

DSPC, cholesterol and PEG2000-DSPE (molar ratio 3:2:0.3) were dissolved in 8 mL chloroform and placed in a 250 mL round-bottomed flask. The solvent was removed by rotary evaporation under reduced pressure at 60° C. Then the resulting dried thin film was hydrated in a 5 mL 250 mM ammoniumsulfate solution (250 mM (NH₄)₂SO₄, pH 5.0, 530 mOsm) and dispersed by hand shaking at 60° C. The resulting suspension of multilamellar vesicles was then frozen and thawed 6 times, followed by repeated extrusion through polycarbonate membrane filters using high-pressure extrusion equipment (Lipex Biomembrane, Vancouver, Canada) at 60° C. The extra-liposomeal salt was removed by gel filtration on Sephadex G-50 column. Doxorubicin stock (10 mg/mL dissolved in ddH₂O) was added immediately into the solution as soon as liposome were eluted from gel filtration column described above at a concentration of 140 g doxorubicin per mole phospholipid. The mixture of liposome and doxorubicin was incubated in a 60° C. water bath with agitation (100 rpm) for 30 minutes. After loading, unencapsulated doxorubicin was removed by Sephadex G-50 gel filtration column equilibrated with 0.9% NaCl solution. The eluted liposome solution was concentrated by ultracentrifugation at 150000×g for 90 minutes. Then resuspend liposome precipitate with 0.9% NaCl solution. Liposomes were sterilized by filtration through 10.22 mm sterile filter and filled into vial B. The quality control of vial B component was as follows:

Vesicles were measured by dynamic laser scattering with a submicron particles analyzer (model N4 plus coulter, Beckman). Particle sizes ranged from 75-95 nm in diameter.

The amount of doxorubicin trapped inside the liposome was determined with a spectrofluorometer (FP6200, JASCO) at an excitaion wavelength of 475 nm and an emission wavelength of 580 nm. Doxorubicin loaded liposomes contained 2 mg doxorubicin per liposome solution.

### EXAMPLE 3

### Preparation of Nano-targeted Liposome Drug in Combined Radionuclide Therapy and Chemotherapy

The vial A containing BMEDA, SnCl₂ and Gluconate-acetate was added with ¹⁸⁸Re solution taken from the vial C and incubated at 80° C. for 1 hour in water bath. The B vial containing Lipo-DXR was added with the ¹⁸⁸Re-BMEDA taken from the vial A solution and incubated at 60° C. for 30 minutes in water bath. The PD-10 column (GE Healthcare) was equilibrated with 20 ml normal saline. The ¹⁸⁸Re-BMEDA Dox-Liposome solution from the B vial was separated from free ¹⁸⁸Re-BMEDA using PD-10 column eluted with normal saline. Each fraction of 0.5 ml was collected into a tube. The red color of Lipo-DXR was used to visually monitor the collection of the ¹⁸⁸Re-BMEDA/DXR-Liposome. The encapsulating efficiency was determined by the quotient of the activity in Lipo-DXR after separation divided by the total activity before separation. The encapsulating efficiency is about 40-60%.

### EXAMPLE 4

### Micro-SPECT Imaging and Images Semi-quantification Analysis of ¹⁸⁸Re-BMEDA-Liposome

Noninvasive in vivo imaging was acquired using low-energy, high-resolution collimators at 1, 4, 24, 48, 72 hr after intravenous injection of ¹⁸⁸Re-BMEDA-Liposomes. The energy window was set at 155 KeV±10˜15%, the image size was set at 64×64, and the radius of rotation (ROR) was 1.0 cm with a FOV (Field of View) of 1.37 cm. The imaging acquisition was accomplished using 64 projection at 120 s per projection. Images were calibrated to standardized uptake values (SUV).

For calculation of standardized uptake value (SUV), a known radio activity Re-188 was performed as reference. The SUV was determined from the regions of interest (ROI) on the tumor with uptake. The SUV was calculated according to the following standard formula:

(measured activity concentration (μCi/g)/[Injected Dose (μCi)/body weight (g)]

The highest SUV of ¹⁸⁸Re-BMEDA-Liposome in tumor by micro-SPECT images semi-quantification analysis was found at 24 h after injection (2.81±0.36), the targeting and nuclear imaging of tumors with the prepared passive nano-targeted Re-188 radionuclides encapsulated in liposomes were demonstrated in FIG. 1.

### EXAMPLE 5

### Therapeutic Efficacy of ¹⁸⁸Re-BMEDA/DXR-Liposome in a C26 Murine Colon Carcinoma Solid Tumor Animal Model

Six-week-old male BALB/c mice were subcutaneously inoculated with 2×105 tumor cells in the right hind flank. The animals were performed for experiments approximately volume with 50˜100 mm³ tumor size after tumor cells inoculation. Tumor bearing mice were divided randomly into 4 groups (I˜IV), eight mice per group, one group was selected randomly as the control. Mice of each group were treated with ¹⁸⁸Re-BMEDA/DXR-Liposome, ¹⁸⁸Re-BMEDA-Liposome, Lipo-DXR and normal saline for 4 groups, respectively, control mice were given equal volumes of normal saline. Tiple-modality treatments were provided in this course. It carried on the treatment of the second time three days after the first treatment and followed by an interval of seven days between the second treatment and third treatment. Mice of group I were received 500 μCi radio-activity in each treatment, group II mice were received 2 mg/kg of doxorubicin and 500 μCi radio-activity and group III mice were only received 2 mg/kg of doxorubicin. Tumors were measured twice weekly to document tumor growth. Tumor measurements were converted to tumor volume (V) using the formula: V=[Y×W2]/2; where Y and W are the larger and smaller perpendicular diameters, respectively. The survival ratio of mice also monitored every day, and the effect of drugs treatment upon the tumor volume were measured twice weekly.

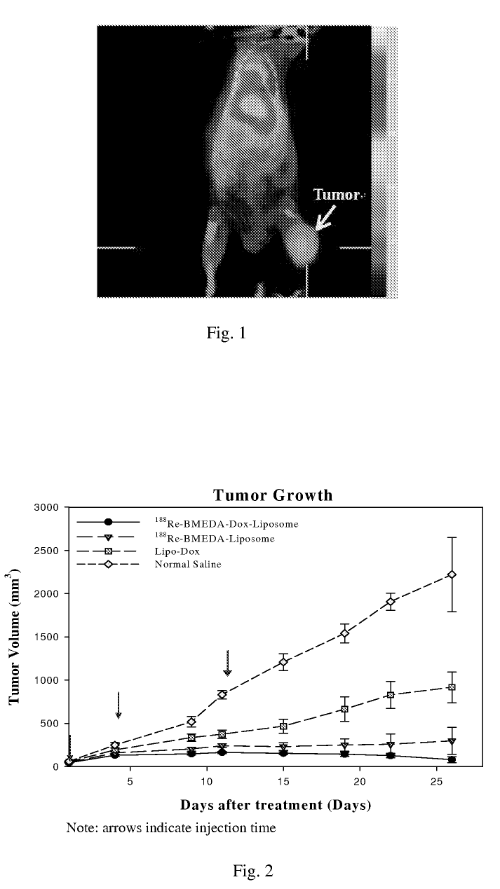

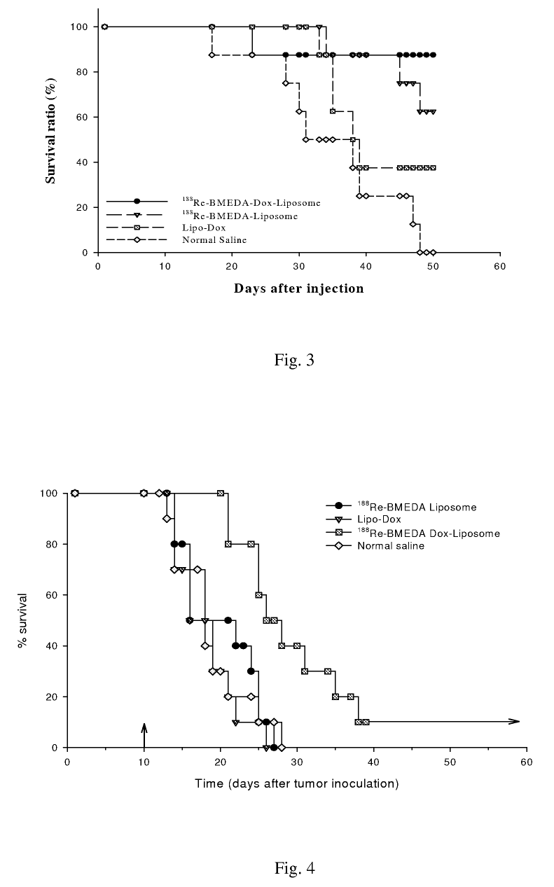

The group of treat with normal saline showed rapid tumor growth, the tumor volume can reach 2220.60±431.35 mm³ at 27 days after administration. The group treated with ¹⁸⁸Re-BMEDA/DXR-Liposome showed smallest tumor volume (80.29±34.94 mm³) than groups of treat with ¹⁸⁸Re-BMEDA-Liposome (298.14±157.25 mm³) and Lipo-DXR (917.20±177.59 mm³) at 27 days after administration. Mice treated with ¹⁸⁸Re-BMEDA/DXR-Liposome showed highest survival ratio (87.5%) than groups of treat with ¹⁸⁸Re-BMEDA-Liposome (62.5%) and Lipo-DXR (37.5%) at 50 days after administration of drugs. The higher therapeutic efficacy of nano-targeted ¹⁸⁸Re-BMEDA/DXR-Liposome and the additive or synergistic effect of bimodality radiochemotherapy on the treatment of C26 solid tumors were demonstrated in FIG. 2 and FIG. 3, respectively.

### EXAMPLE 6

### Therapeutic Evaluation of ¹⁸⁸Re-BMEDA/DXR-Liposome in Malignant Carcinoma Ascites Mouse Model (Intraperitoneal Injection)

Male BALB/c mice (6 weeks old) were inoculated intraperitoneal (i.p.) with 2×105 C26 cells in 500 μl PBS. After 10 days, abdominal swelling developed, indicating peritoneal tumor spread and producing ascites. For therapeutic evaluation, mice were treated at 10 days after tumor cell inoculation.

A total of forty BALB/c mice were divided into 4 groups (n=10) after i.p. inoculation with 2.5×105 C26 cells. Group A received i.p. injection of 200 μl ¹⁸⁸Re-BMEDA Liposome containing 22.2 MBq of radioactivity and 0.93 μmole phospholipid. Group B received i.p. injection of 5 mg/kg Lipo-DXR dissolved in 200 μl normal saline containing 0.128 mg of doxorubicin and 0.93 μmole phospholipid. Group C received i.p. injection of 200 μl ¹⁸⁸Re-BMEDA/DXR-Liposome containing 22.2 MBq of radioactivity, 0.128 mg of doxorubicin (5 mg/kg) and 0.93 μmole phospholipid. Group D received i.p. injection of 200 μl normal saline. The mice were checked survival and body weight every day until death. The experiment endpoint was terminated at 120 d after tumor cell inoculation. The remaining mice were euthanized and dissected for observed whether tumor nodules were remained.

The table 2 shows that the median survival time of the mice treated with ¹⁸⁸Re-BMEDA Liposome, Lipo-DXR, ¹⁸⁸Re-BMEDA/DXR-Liposome and normal saline were 21 d, 18 d, 27 d (P<0.05) and 17.67 d, respectively (P values, determined by use of the log-rank test, were for comparisons with mice receiving normal saline).

TABLE 2

| Group | Median survival time | |
|---|---|---|
| (d) | (Compared with NS) | P value |
| ¹⁸⁸Re-BMEDA | 21 | 0.8715 |
| liposomes | | |
| Lipo-DXR | 18 | 0.7558 |
| ¹⁸⁸Re-BMEDA/ | 27 | 0.0014 |
| DXR-Liposomes | | |
| Normal saline (NS) | 17.67 | |

Note:
1. P values were determined by used of log-rank test.
2. The level of statistical significance was set at a P value of <0.05.

The FIG. 4 shows that the mice receiving i.p. injection of ¹⁸⁸Re-BMEDA/DXR-Liposome were survival of 10% mice. Other groups were no remaining mice. The results demonstrated the additive or synergistic effectiveness of bimodality radiochemotherapy of ¹⁸⁸Re-BMEDA /DXR-Liposome on murine C26 ascites animal model with i.p injection.

What is claimed is:

1. A kit composition, comprising:
(i) a 10 ml vial A which contains BMEDA, gluconate acetate, SnCl₂; (ii) a 10 ml vial B which contains DSPC, cholesterol, DSPE-PEG, and chemotherapy drug;
(iii) a 10 ml vial C which contains radionuclide solution.

2. The kit composition according to claim 1, wherein said chemotherapy drug is Doxorubicin or Daunorubicin or Vinolbine.

3. The kit composition according to claim 1, wherein said radionuclide solution is ¹⁸⁸Re or ¹⁸⁶Re.

4. A process for using the kit of claim 1, comprising the steps of:
(i) Remove contents of the radionuclide solution from vial C; (ii) Inject the radionuclide solution into the vial A to produce mixtures, and the mixtures react in appropriate temperature; (iii) Remove contents of radionuclide from vial A; (iv) Inject the radionuclide solution into the vial B to produce mixtures, and the mixtures react in appropriate temperature; and (v) a reconstituted solution is obtained in the vial B.

5. The process according to claim 4, wherein said radionuclide solution is ¹⁸⁸Re or ¹⁸⁶Re.

6. The process according to claim 4, wherein said radionuclide solution is ¹⁸⁸Re-BMEDA or ¹⁸⁶Re-BMEDA.

7. The process according to claim 4, wherein said appropriate temperature is 40° C.˜110° C.

8. The process according to claim 4, wherein said reconstituted solution is ¹⁸⁸Re-BMEDA/DXR-Liposome or ¹⁸⁶Re-BMEDA/DXR-Liposome.

9. A reconstituted solution in the process of claim 4 is applicable to combine targeted radionuclide therapy and chemotherapy for imaging and treatment of tumor and malignant ascites.
